(19) United States
(12) Patent Application Publication
(10) Pub. No.: US 2026/0013912 A1
(43) Pub. Date: Jan. 15, 2026

(54) SYSTEM AND METHODS FOR BONE TRANSPORT

(71) Applicant: NuVasive Specialized Orthopedics, INC., San Diego, CA (US)

(72) Inventors: Jeffrey Schwardt, San Diego, CA (US); Michael Moeller, San Diego, CA (US); Thomas B. Buford, San Diego, CA (US); Vijayendran Somasegaran, San Diego, CA (US)

(21) Appl. No.: 19/332,547

(22) Filed: Sep. 18, 2025

Related U.S. Application Data
(63) Continuation of application No. 17/136,993, filed on Dec. 29, 2020.
Application No. 17/136,993 is a continuation of application No. 16/046,909, filed on Jul. 26, 2018, now Pat. No. 10,918,425.

(51) Int. Cl. A61B 17/72; A61B 17/17; A61B 17/62; A61B 17/64; A61B 17/66; A61B 17/68; A61B 17/80; A61B 17/88

(52) U.S. Cl. CPC A61B 17/7216; A61B 17/62; A61B 17/66; A61B 17/8004; A61B 17/8019; A61B 17/171; A61B 17/64; A61B 2017/681; A61B 17/8866; A61L 2430/02

(57) ABSTRACT

A system for bone transport is provided, the system comprising: an adjustable length implant configured for intramedullary placement and comprising a first end configured to be coupled to bone and a second end configured to be coupled to bone, wherein the first end and the second end are displaceable relative to each other along a longitudinal axis; and a driving element configured to be non-invasively activated to displace the first and second ends relative to one another along the longitudinal axis; and a support member having distal and proximal ends, wherein the support member includes a longitudinally extending slot disposed between the distal and proximal ends of the support member, the slot having opposing ends, wherein the slot is configured to pass an elongate anchor such that the elongate anchor is slidable between the first end and the second end of the slot.

### CROSS REFERENCE TO RELATED APPLICATIONS

The present application is a continuation of U.S. patent application Ser. No. 17/136,993, filed on Dec. 29, 2020 (published as U.S. Pat. Pub. No. 2021-0113247), which is a continuation of U.S. patent application Ser. No. 16/046,909, filed on Jul. 26, 2018, now U.S. Pat. No. 10,918,425, which is a continuation of PCT/US17/15555 filed Jan. 30, 2017, which claims the benefit of priority of U.S. Provisional Application No. 62/288,348, filed Jan. 28, 2016. Each of these applications is hereby incorporated-by-reference in its entirety for all purposes.

### BACKGROUND

Distraction osteogenesis is a technique which has been used to grow new bone in patients with a variety of defects. For example, limb lengthening is a technique in which the length of a bone (for example a femur or tibia) may be increased. After creating a corticotomy, or osteotomy, in the bone, which is a cut through the bone, the two resulting sections of bone may be moved apart at a particular rate, such as one (1.0) mm per day. New bone may regenerate between the two sections of the bone as they are moved apart. This technique of limb lengthening can be used in cases in which one limb is longer than the other, such as in a patient whose prior bone break did not heal correctly, or in a patient whose growth plate was diseased or damaged prior to maturity. In some patients, stature lengthening is desired and may be achieved by lengthening both femurs and/or both tibiae to increase the patient's height.

Bone transport is a similar procedure, in that it makes use of osteogenesis. But, instead of increasing the distance between the ends of a bone, bone transport fills in missing bone in between. There are several reasons why significant amounts of bone may be missing. For example, a prior non-union of bone, such as that from a fracture, may have become infected necessitating removal of the infected section. Also, segmental defects may be present, the defects often occurring from severe trauma when large portions of bone are severely damaged. Other types of bone infections or osteosarcoma may require removal of a large piece of bone (causing a portion of the natural bone to be missing).

Historically, limb lengthening was often performed using external fixation. The external fixation process involves an external distraction frame which may be attached to two (or more) separate sections of bone by transdermal pins (i.e., passing through the skin). Pin-based methods suffer from several shortcomings. For example, the pins can be sites for infection and are often painful for the patient, as the pin placement site remains a somewhat open wound “pin tract” throughout the treatment process. External fixation frames are also bulky, and can make it difficult for the patient to comfortably sit, sleep, and move. Intramedullary lengthening devices also exist, such as those described in U.S. patent application Ser. No. 12/875,585, which is incorporated by reference herein.

Bone transport is frequently performed by either external fixation, or by bone grafting. In external fixation bone transport, a bone segment is cut from the remaining sections of bone and moved by the external fixation, usually at a rate close to one (1.0) mm per day, until the resulting regenerate bone fills the defect. The wounds created from the pin tracts in external fixation-based bone transport procedures are frequently even worse than those created by external fixation limb lengthening procedures. The pins begin to open the wounds larger as the pins are moved with respect to the skin. In bone grafting, autograft (from the patient) or allograft (from another person) is typically used to create a lattice for new bone growth. Bone grafting can be more complicated and/or expensive than the placement of external fixation pins.

### SUMMARY

The present disclosure provides for a method for transporting a portion of bone within a patient having an incomplete bone including providing an adjustable-length implant configured for intramedullary placement and having a first end configured to be coupled to bone and a second end configured to be coupled to bone, wherein the first end and the second end are displaceable relative to each other along a longitudinal axis, placing the adjustable-length implant at least partially within the medullary canal of a bone of a subject, the bone having first and second ends and having at least first and second portions having a space there between, the first portion of the bone including the first end of the bone and the second portion of the bone including the second end of the bone, creating a third portion of the bone by detaching at least some of either the first portion of the bone or the second portion of the bone, wherein the third portion of the bone does not include the first end of the bone or the second end of the bone, coupling a support member having first and second ends to the bone by coupling the first end of the support member to an external surface of the first portion of the bone and coupling the second end of the support member to an external surface of the second portion of the bone, coupling the first end of the adjustable-length implant to one of the first and second portions of the bone, coupling the second end of the adjustable-length implant to the third portion of the bone, wherein the adjustable-length implant includes a driving element configured to be non-invasively activated such that a distance between the first end and the second end of the adjustable-length implant is controllably changed such that the third portion of the bone is moved along the longitudinal axis in relation to the first and second portions of the bone, while the first portion of the bone and second portion of the bone are not moved in relation to each other.

The present disclosure additionally provides for a system for bone transport including an adjustable length implant configured for intramedullary placement and having a first end configured to be coupled to bone and a second end configured to be coupled to bone, wherein the first end and the second end are displaceable relative to each other along a longitudinal axis, and a driving element configured to be non-invasively activated such that a distance between the first end and the second end of the adjustable-length implant can be controllably along the longitudinal axis, and a support member having first and second ends, wherein the support member includes a longitudinally extending slot disposed between the first and second ends of the support member, the slot having a first end and a second end, wherein the slot is configured to pass an elongate anchor such that the elongate anchor is slidable between the first end and the second end of the slot.

The present disclosure further provides for a method for transporting a portion of bone within a patient having an incomplete bone including providing an adjustable-length implant configured for intramedullary placement and having a first end configured to be coupled to bone and a second end configured to be coupled to bone, wherein the first end and the second end are displaceable relative to each other along a longitudinal axis, placing the adjustable-length implant at least partially within the medullary canal of a bone of a subject, the bone having first and second ends and having at least first and second portions having a space there between, the first portion of the bone including the first end of the bone and the second portion of the bone including the second end of the bone, creating a third portion of the bone by detaching at least some of either the first portion of the bone or the second portion of the bone, wherein the third portion of the bone does not include the first end of the bone or the second end of the bone, coupling an external fixator to the bone, the external fixator having an external base, a first pin and a second pin, by coupling the first pin of the external fixator to the first portion of the bone and coupling the second pin of the external fixator to the second portion of the bone, coupling the second end of the adjustable-length implant to the third portion of the bone, wherein the adjustable-length implant includes a driving element configured to be non-invasively activated such that a distance between the first end and the second end of the adjustable-length implant is controllably changed such that the third portion of the bone is moved along the longitudinal axis in relation to the first and second portions of the bone, while the first portion of the bone and second portion of the bone are not moved in relation to each other.

### BRIEF DESCRIPTION OF THE DRAWINGS

FIGS. 1-2 illustrate various views of an intramedullary device configured for bone transport.

FIG. 3 illustrates a sectional view of the intramedullary device of FIG. 2 taken along line 3-3.

FIG. 4A illustrates detailed view 4 of FIG. 3.

FIG. 4B illustrates a sectional view of another embodiment of an intramedullary device.

FIG. 4C illustrates a ring gear insert of the device shown in FIG. 4B.

FIG. 4D illustrates a coupling assembly of the device shown in FIG. 4B.

FIG. 5 illustrates an exploded view of the intramedullary device shown in FIGS. 1-4A.

FIG. 6 illustrates detailed view 6 of FIG. 5.

FIG. 7 illustrates a sectional view of another embodiment of an intramedullary device.

FIG. 8 illustrates a maintenance member of the intramedullary device of FIG. 7.

FIGS. 9-12 schematically illustrate various driving elements of an intramedullary device.

FIG. 13 illustrates a bone with a portion missing.

FIG. 14 illustrates a system for bone transport coupled to a bone.

FIG. 15 illustrates the system of FIG. 14 after the transport of a portion of bone.

FIG. 16 illustrates a system for bone transport coupled to a bone in a retrograde manner.

FIG. 17 illustrates the system of FIG. 16 after the transport of a portion of bone.

FIG. 18 illustrates an external adjustment device.

FIG. 19 illustrates an exploded view of a magnetic hand piece of the external adjustment device of FIG. 18.

FIG. 20 illustrates another embodiment of a system for bone transport.

FIG. 21 illustrates the system of FIG. 20 coupled to a bone.

FIG. 22 illustrates the system of FIG. 21 after the transport of a portion of bone.

FIG. 23 illustrates a kit for an adjustable-length implant.

FIG. 24 illustrates an adjustable-length implant constructed from the kit of FIG. 23.

### DETAILED DESCRIPTION

Various adjustable devices for implanting into the body that are capable of changing or working/acting on a portion of the skeletal system of a patient are disclosed herein. In some embodiments, the adjustable implants are configured for transporting a segment of bone to replace lost portions of bone. Methods for using the adjustable implants for transporting a segment of bone in order to replace lost portions of bone are also provided. In some embodiments, the method may incorporate one or more plates. Adjustable devices may include distraction or retraction devices, for example, distraction or retraction devices configured for orthopedic applications, including, but not limited to scoliosis, limb lengthening, bone transport, spinous process distraction, lumbar lordosis adjustment, tibial wedge osteotomy adjustment, and spondylolisthesis. Adjustable devices configured for bone transport may include intramedullary limb lengthening devices.

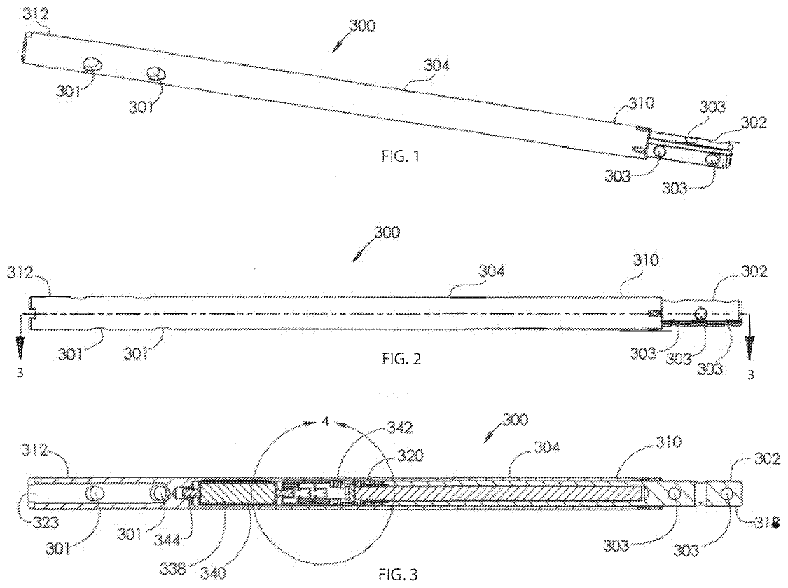

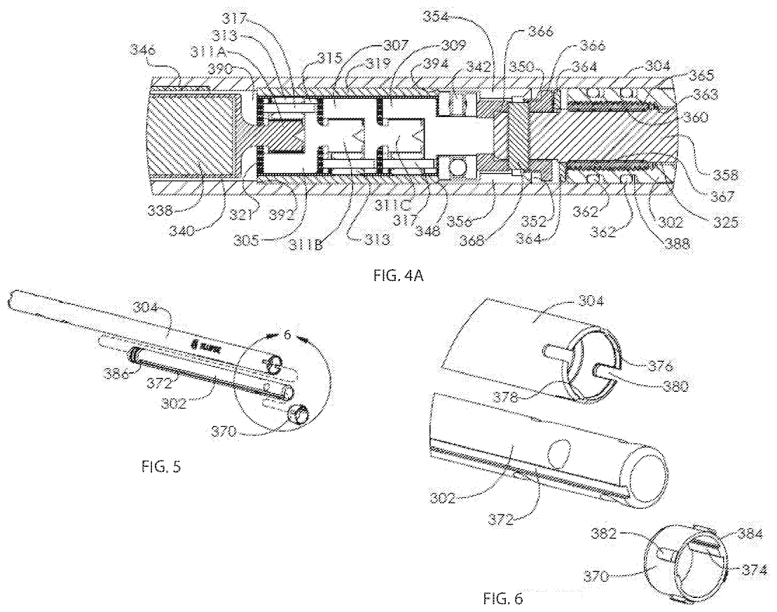

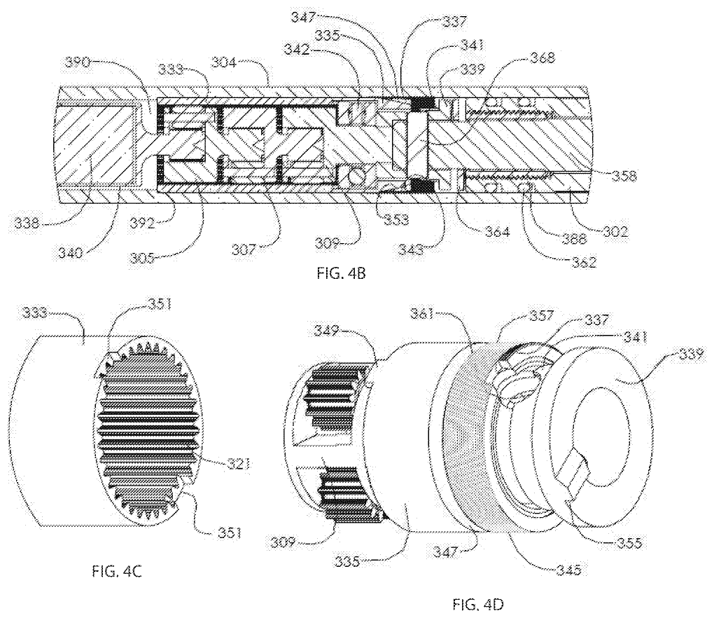

FIGS. 1 and 2 illustrate an intramedullary device 300 (e.g., an intramedullary lengthening device) comprising a distraction rod 302 and a housing 304. The housing 304 extends between a first end 310 and a second end 312, as may be better appreciated in the sectional view of FIG. 3. The housing 304 may be formed as a unitary structure with no seams or joints. Alternatively, the housing 304 may be formed in pieces that are fused together at seams or joints. As shown in FIG. 3, the distraction rod 302 has a first end 318 and a second end 320, and is configured to be telescopically extendable and retractable relative to the housing 304 (e.g., within the housing 304). Like the housing 304, the distraction rod 302 may be a unitary structure with no seams or joints connecting various sub-components. Alternatively, the distraction rod 302 may be formed in pieces that are fused together at seams or joints. Both the distraction rod 302 and the housing 304 may be made from any of a number of biocompatible materials, including titanium, for example Titanium-6AL-4V, cobalt chromium alloys, and stainless steel. Because the distraction rod 302 and the housing 304 are the primary load bearing members of the intramedullary device 300, and because neither has any external circumferential weld(s), the intramedullary device 300 can be capable of withstanding improved loading challenges in comparison to conventional intramedullary limb lengthening devices. The housing 304 contains at least one transverse hole (e.g., two transverse holes 301) for passing bone screws, with which to attach the intramedullary device 300 to the bone. The distraction rod 302 contains at least one transverse hole (e.g., three transverse holes 303), also for the passing of bone screws. As will be readily understood, the number and orientation of the transverse holes 301, 303 may be varied as necessary, useful, or desired for any given application. At the second end 312 of the housing 304, a coupling feature 323, provides an interface to releasably engage with an insertion instrument, such as a drill guide. The drill guide may include a male thread and the coupling feature 323 may have a complementary or mating female thread. The intramedullary device 300 comprises a magnet 338 which is bonded within a magnet housing 340 and configured for rotation between a radial bearing 344 and a thrust bearing 342 (shown more clearly in FIG. 4A). Between the thrust bearing 342 and the magnet housing 340 is at least one planetary gear stage (e.g., three planetary gear stages 305, 307, 309, as seen in FIG. 4A). Each planetary gear stage (e.g., planetary gear stages 305, 307, 309) comprises a sun gear (e.g., sun gear 311A, 311B, 311C) and a plurality of planetary gears (e.g., three planetary 313), which are rotatably held within a frame 315 by pins 317. The sun gear 311 is either a part of the magnet housing 340, as in the case of the sun gear 311A of planetary gear stage 305, or a part of the frame 315, as in sun gear 311B of gear stage 307 and sun gear 311C of gear stage 309. The rotation of the sun gear 311 causes the planetary gears 313 to rotate and track along inner teeth 321 of a ring gear insert 319. Each gear stage has a gear reduction ratio (e.g., of 4:1), which results in a total gear reduction (e.g., a total gear reduction of 64:1-provided by three planetary gear stages each having a reduction ratio of 4:1). It should be understood that other gear reductions, and numbers of stages may be used.

The frame 315 of the final gear stage (e.g., gear stage 309) passes through the thrust bearing 342 and is attached to a lead screw coupler 366 such that rotation of the frame 315 of the final gear stage 309 causes one-to-one rotation of the lead screw coupler 366. The lead screw coupler 366 and a lead screw 358 each contain transverse holes through which a locking pin 368 is placed, thus rotationally coupling the lead screw 358 to the final gear stage (e.g., gear stage 309). A locking pin retainer 350 is slid over and secured (e.g., tack welded) to the lead screw coupler 366 to radially maintain/retain the locking pin 368 in place. The distraction rod 302 has an internally threaded end 363, into which external threads 365 of a nut 360 are threaded and bonded, for example with epoxy. The nut 360 has internal threads 367 which are configured to threadably engage with external threads 325 of the lead screw 358, thereby allowing rotation of the lead screw 358 in a first direction to distract or extend the distraction rod 302 in relation to the housing 304. Rotation of the lead screw 358 in a second (opposite) direction retracts or withdraws the distraction rod 302 in relation to the housing 304. Rotation of the magnet 338 and the magnet housing 340 causes rotation of the lead screw. Depending on the gearing included, rotation of the magnet 338 and the magnet housing 340 can cause rotation of the lead screw 358 at 1/64 the rotational speed, but with significantly increased torque (64 times, minus frictional losses), and thus an amplified distraction or extension force. O-rings 362 are placed in ring grooves 388 on the exterior of the distraction rod 302 to create a dynamic seal between the housing 304 and the distraction rod 302 that protects the internal contents from body fluids. A split washer stop 364, located between the distraction rod 302 and the lead screw coupler 366, guards against jamming that could otherwise be caused as the distraction rod 302 approaches the lead screw coupler 366, for example if intramedullary device 300 is fully retracted with a high torque (e.g., a high torque applied by an external moving magnetic field).

A maintenance member 346, comprising a curved plate made from a magnetically permeable material (e.g., 400 series stainless steel), is secured to/bonded within the inner wall of the housing 304 (e.g., using epoxy, adhesive, resistance welding, or other suitable process(es)). The maintenance member 346 attracts a pole of the magnet 338, thus keeping the limb lengthening device 300 from being accidentally adjusted by movements of the patient. However, a strong moving magnetic field, such as that applied by magnetic adjustment devices known in the art, is capable of overcoming the attraction of the magnet 338 to the maintenance member 346, rotate the magnet 338, and thereby adjust the length of the intramedullary device 300. The maintenance member 346 can have has a thickness of approximately 0.015 inches and can span a circumferential arc of less than about 180° (e.g., an exemplary arc is) 99°. Of course, other dimensions for the maintenance member 346 are contemplated, as long as it provides sufficient attractive force(s) to the magnet 338 to appropriately hold it in place when not being actuated.

The distraction rod 302 and the housing 304 may be individually manufactured, for example by machining processes incorporating manual or automated lathes. Included within this manufacturing operation may be the forming of an axially-extending cavity within the housing 304. Post-processing may be included in this operation, for example bead blasting, passivation, and/or anodizing. The distraction rod 302 and the housing 304 are then prepared for mating. In this operation, the nut 360 is bonded into the distraction rod 302 and the O-rings 362 are placed into the ring grooves 388 as described. The maintenance member 346 is bonded to the housing 304. Then, the magnet 338 is placed into the cavity 390 of the housing 304. In this operation the magnet 338 and the magnet housing 340 are bonded together, and then assembled with the radial bearing 344 into the housing 304 (see FIG. 3). Prior to assembling the radial bearing 344 into the housing 304, the longitudinal depth of the cavity 390 of the housing 304 is measured, and, if necessary, one or more shims may be placed before the radial bearing 344. Ideally, the axial play in the assembled components is not so low as to cause binding, yet not so high as to risk disassembly. Next, the lead screw 358 is prepared for coupling to the magnet 338 that is in the cavity 390 of the housing 304. In this operation the ring gear insert 319 is slid into the cavity 390 of the housing 304 until it abuts ledge 392. First and second planetary gear stages 305, 307 are then placed into the assembly as seen in FIG. 4A. The locking pin retainer 350 is preloaded over the lead screw coupler 366 prior to welding the lead screw coupler 366 to the final planetary gear stage 309, and is then slid in place over the locking pin 368 after the locking pin 368 is placed. Final planetary gear stage 309 is inserted through the thrust bearing 342 and is welded to the lead screw coupler 366, allowing for some axial play of the thrust bearing 342. The split washer stop 364 is then placed onto the lead screw 358. The lead screw 358 is then attached to the lead screw coupler 366 with the locking pin 368, and then the locking pin retainer 350 is slid over a portion of the ends of the locking pin 368 and tack welded to the lead screw coupler 366. Thrust bearing retainers 354, 356 are two matching pieces which form a cylindrical clamshell around the thrust bearing 342 and the lead screw coupler 366. The internal diameter of the housing 304 is tinned with solder, as are the outer half diameter surfaces of each of the thrust bearing retainers 354, 356. Next, the thrust bearing retainers 354, 356 are clamped over an assembly comprising the thrust bearing 342, lead screw coupler 366, planetary gear stage 309, and lead screw 358, and the thrust bearing retainers 354, 356 are pushed together into place within the housing 304, for example with the aid of a tool pressed against chamfers 352 of the thrust bearing retainers 354, 356. The sun gear 311C of the final planetary gear stage 309 engages with the planet gears 317 of the final planetary gear stage 309 and then chamfered edges 394 of the thrust bearing retainers 354, 356 are pushed against a chamfer 348 of the ring gear insert 319 and a compressive force is held. Next, the thrust bearing 342 and the magnet 338 are axially retained. In this operation, the thrust bearing retainers 354, 356 are soldered to the housing 304 at the tinned portions, thus maintaining compressive force. This may be accomplished using induction heating. The friction of the ledge 392 and the chamfered edge 394 against opposing ends of the ring gear insert 319, as well as the wedging between the chamfered edge 394 and the chamfer 348, create a resistance to rotation, thus holding the ring gear insert 319 rotationally static in relation to the housing 304. Alternatively, the ring gear insert 319 may have a keyed feature that fits into a corresponding keyed feature in the housing 304, in order to stop the ring gear insert 319 from turning relative to the housing 304 (this may be useful if/when the friction on the ends of the ring gear insert 319 is not sufficient to hold the ring gear insert 319 static).

The distraction rod 302 can then be engaged with the lead screw 358. In this operation, an assembly tool, such as a high speed rotating magnet, is used to make the magnet 338 and, consequently, the lead screw 358 rotate and the distraction rod 302 is inserted into the housing 304 while the lead screw 358 engages and displaces with respect to the nut 360 of the distraction rod 302. After the distraction rod 302 is inserted into the housing 304 as described and retracted at least somewhat, the distraction rod 302 is still free to rotate with respect to the housing 304. For the stability of the bone pieces being distracted, it may be desirable to inhibit rotation between the distraction rod 302 and the housing 304. One possible method and structure of doing so is described in relation to FIGS. 5 and 6. The distraction rod 302 may be rotationally locked with respect to the housing 304 by placing an anti-rotation ring 370 over the distraction rod 302 by engaging protrusions 374, one on each side, into grooves 372 extending along the distraction rod 302 and then by sliding the anti-rotation ring 370 up to a tapered inner edge 376 of the housing 304. The anti-rotation ring 370 and the distraction rod 302 may then be rotated until guide fins 382 can be inserted (e.g., slide) into guide cuts 380 in the end of the housing 304. The anti-rotation ring 370 can be axially snapped into the housing 304 so that flat edge 384 of the anti-rotation ring 370 is trapped by undercut 378. The undercut 378 has a minimum diameter which is less than the outer diameter of the flat edge 384 of the anti-rotation ring 370, and is temporarily forced open during the snapping process. As assembled, the anti-rotation ring 370, the housing 304 and the distraction rod 302 are all held substantially rotationally static in relation to each other. In addition, when the intramedullary device 300 reaches its maximum distraction length, the ends 386 of grooves 372 abut the protrusions 374, thereby keeping the distraction rod 302 from falling out of the housing 304.

An alternative embodiment of the intramedullary device 300 of FIGS. 1-4A is shown in a sectional view in FIG. 4B. Much of this embodiment can be similar or identical to the embodiments shown in FIGS. 1-4A. However, this embodiment varies at least in that it need not have thrust bearing retainers 354, 356. Instead, it may incorporate a thrust bearing ferrule having an external tapered end 347. A thrust bearing retainer 337, a locking pin retainer 341, and the thrust bearing ferrule 335 are placed over the thrust bearing 342 and a lead screw coupler 339 and the final planetary gear stage 309 are inserted through the thrust bearing 342 and welded to the lead screw coupler 339. As shown in FIG. 4D, the locking pin retainer 341 has a relief 361 to allow the passage of the locking pin 368. After the locking pin 368 is placed, the locking pin retainer 341 may be rotated so that the relief 361 is no longer directly over the locking pin 368 and the locking pin retainer 341 is tack welded or secured by other methods to the lead screw coupler 339, thus retaining the locking pin 368. These assembled components are then inserted into the cavity 390 of the housing 304, where the final planetary gear stage 309 is coupled to the other planetary gear stages 305, 307 and the magnet 338. In this embodiment, a ring gear insert 333 (FIG. 4C) has an indentation 351 (e.g., a notch) on each side. A tab 349 on each side of the thrust bearing ferrule 335 inserts into each indentation 351 and inhibits rotation of the ring gear insert 333 in relation to the housing 304 once the thrust bearing ferrule 335 is engaged into the housing 304. Also in this embodiment, the housing 304 contains internal threading 343. The engagement of the thrust bearing ferrule 335 is achieved by tightening external threading 345 of the thrust bearing retainer 337 into the internal threading 343 of the housing 304. A tool (not shown) may be engaged into cut outs 357 on either or both sides of the thrust bearing retainer 337 and is used to screw the thrust bearing retainer 337 into the internal threading 343 of the housing 304. As shown in FIG. 4B, this wedges an internal taper 353 of the thrust bearing retainer 337 against the external tapered end 347 of the thrust bearing ferrule 335, allowing the thrust bearing ferrule 335 to apply a controlled load on the ring gear insert 333, locking the ring gear insert 333 axially and rotationally with respect to the housing 304. The thrust bearing retainer 337 contains an axial split on the opposite side (not shown). The split in the thrust bearing retainer 337, allows the outer diameter of the thrust bearing retainer 337 to be slightly reduced (by compression) while it is inserted into the housing 304, prior to being threaded, so that the internal portion of the housing 304 is not scratched during insertion. A ledge 355 is visible on the lead screw coupler 339 in FIG. 4D. As noted earlier, the split washer stop 364 butts up against this ledge 355 to prohibit jamming when the distraction rod 302 is retracted completely.

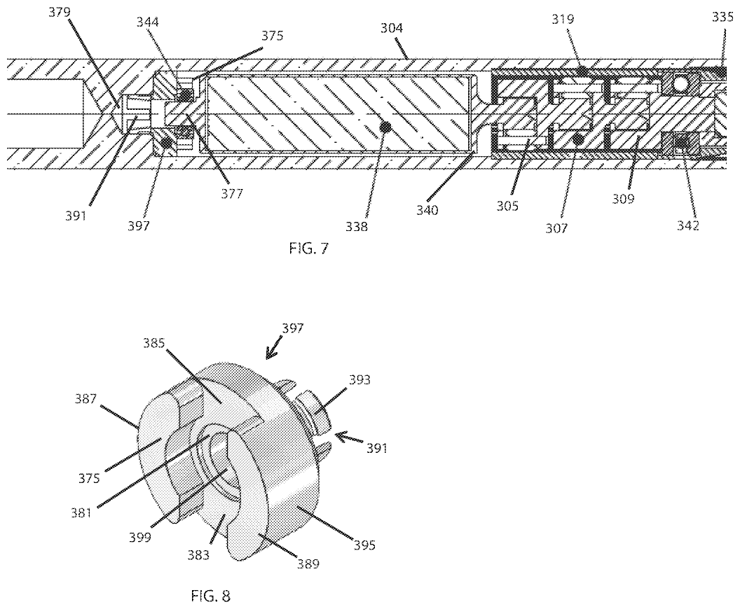

An alternative embodiment of the intramedullary device 300 of FIGS. 1-4A is shown in a sectional view in FIG. 7. A maintenance member 397 replaces the curved plate maintenance member 346. The maintenance member 397 is spaced axially in relation to the magnet 338 within the housing 304 of the limb lengthening device 300, but because of its proximity to the magnet 338, maintenance member 397 is still capable of attracting a pole of the magnet 338, thus keeping the limb lengthening device 300 from being accidentally adjusted by movements of the patient. The maintenance member 397 comprises a body 395 and a securement portion 391. The securement portion 391 is illustrated as comprising four tabs 393, each having an outer radius that is greater than the radius of cavity 379 in the housing 304. The interference between the tabs 393 and the cavity 379 is sufficient to hold the maintenance member 379 in place, so that it cannot turn or move axially in relation to the housing 304. Alternatively, the securement portion 391 may be adhesively bonded, welded, or secured by another means to the cavity 379. The maintenance member 397 includes a ledge 381 which is configured to seat the radial bearing 344. Similar to the embodiments of FIGS. 1-4D, a nose 377 of the magnet housing 340 is pressed into the inner hole of the radial bearing 344. In the embodiment of FIGS. 7 and 8, a through hole 399 in the maintenance member 397 is configured to allow non-contact extension of the nose 377 of the magnet housing 340, thus allowing the magnet housing 340, and thus magnet 338, to freely rotate. Ears 387, 389 are separated by gaps 383, 385, and comprise a magnetically permeable material (e.g., 400 series stainless steel, iron, mu-metal, or another similar material that can attract a pole of the magnet 338). An edge 375 of each ear 387, 389 may be flat, in order to allow a maximal amount of material to be located in proximity to the magnet 338.

FIG. 18 illustrates an external adjustment device 1180 that is used to non-invasively adjust the devices and systems described herein. The external adjustment device 1180 comprises a magnetic hand piece 1178, a control box 1176 and a power supply 1174. The control box 1176 includes a control panel 1182 having one or more controls (buttons, switches or tactile, motion, audio or light sensors) and a display 1184. The display 1184 may be visual, auditory, tactile, the like or some combination of the aforementioned features. The external adjustment device 180 may contain software that allows programming by the physician.

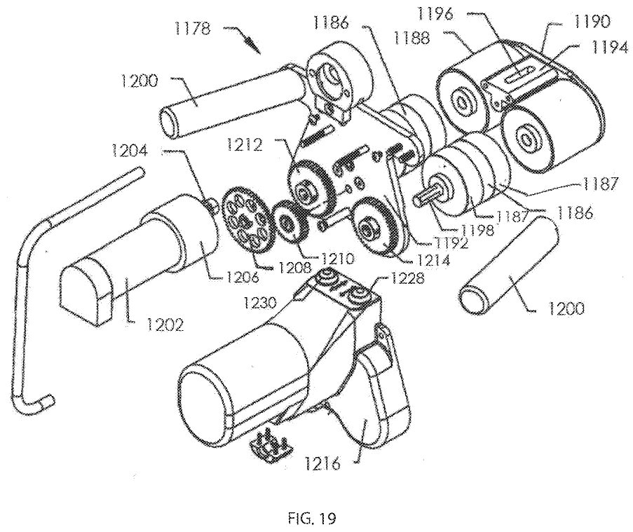

FIG. 19 shows the detail of the magnetic hand piece 1178 of the external adjustment device 1180. There is a plurality of, e.g., two (2), magnets 1186 that have a cylindrical shape (also, other shapes are possible). In some embodiments, the magnetic hand piece 1178 comprises only one magnet 1186. In some embodiments, the magnetic hand piece 1178 uses one or more electromagnets. The magnets 1186 can be made from rare earth magnets (such as Neodymium-Iron-Boron), and can in some embodiments be radially poled. The magnets 1186 are bonded or otherwise secured within magnetic cups 1187. The magnetic cups 1187 each include a shaft 1198, one of which is attached to a first magnet gear 1212 and the other of which is attached to a second magnet gear 1214. The orientation of the poles of each the two magnets 1186 are maintained in relation to each other by means of the gearing system (by use of center gear 1210, that meshes with both first magnet gear 1212 and second magnet gear 1214). In one embodiment, the north pole of one of the magnets 1186 turns synchronously with the south pole of the other magnet 1186, at matching clock positions throughout a complete rotation. The configuration has been known to provide an improved delivery of torque, for example to magnet 338. Examples of methods and embodiments of external adjustment devices that may be used to adjust the intramedullary device 300, or other embodiments of the present invention, are described in U.S. Pat. No. 8,382,756, and U.S. patent application Ser. No. 13/172,598, both of which are incorporated by reference herein.

The components of the magnetic hand piece 1178 are held together between a magnet plate 1190 and a front plate 1192. Most of the components are protected by a cover 1216. The magnets 1186 rotate within a static magnet cover 1188, so that the magnetic hand piece 1178 may be rested directly on the patient, while not imparting any motion to the external surfaces of the patient. Prior to distracting the intramedullary lengthening device 1110, the operator places the magnetic hand piece 1178 over the patient near the location of the magnet 338. A magnet standoff 1194 that is interposed between the two magnets 1186 contains a viewing window 1196, to aid in the placement. For instance, a mark made on the patient's skin at the appropriate location with an indelible marker may be viewed through the viewing window 1196. To perform a distraction, the operator holds the magnetic hand piece 1178 by its handles 1200 and depresses a distract switch 1228, causing motor 1202 to drive in a first direction. The motor 1202 has a gear box 1206 which causes the rotational speed of an output gear 1204 to be different from the rotational speed of the motor 1202 (for example, a slower speed). The output gear 1204 then turns a reduction gear 1208 which meshes with center gear 1210, causing it to turn at a different rotational speed than the reduction gear 1208. The center gear 1210 meshes with both the first magnet gear 1212 and the second magnet gear 1214 turning them each at the same rate. Depending on the portion of the body where the magnets 1186 of the external adjustment device 1180 are located, it is desired that this rate be controlled, to minimize the resulting induced current density imparted by magnet 1186 and magnet 338 through the tissues and fluids of the body. For example a magnet rotational speed of 60 RPM or less is contemplated although other speeds may be used such as 35 RPM or less. At any time, the distraction may be lessened by depressing the retract switch 1230, which can be desirable if the patient feels significant pain, or numbness in the area holding the device.

Throughout the embodiments presented, a magnet 338 is used as a driving element to remotely create movement in an intramedullary device 300. FIGS. 9-12 schematically show four alternate embodiments, wherein other types of energy transfer are used in place of permanent magnets.

FIG. 9 illustrates an intramedullary device 1300 comprising an implant 1306 having a first implant portion 1302 and a second implant portion 1304, the second implant portion 1304 being non-invasively displaceable with respect to the first implant portion 1302. The first implant portion 1302 is secured to a first bone portion 197 and the second implant portion 1304 is secured to a second bone portion 199 within a patient 191. A motor 1308 is operable to cause the first implant portion 1302 and the second implant portion 1304 to displace relative to one another. An external adjustment device 1310 has a control panel 1312 for input by an operator, a display 1314 and a transmitter 1316. The transmitter 1316 sends a control signal 1318 through the skin 195 of the patient 191 to an implanted receiver 1320. Implanted receiver 1320 communicates with the motor 1308 via a conductor 1322. The motor 1308 may be powered by an implantable battery, or may be powered or charged by inductive coupling.

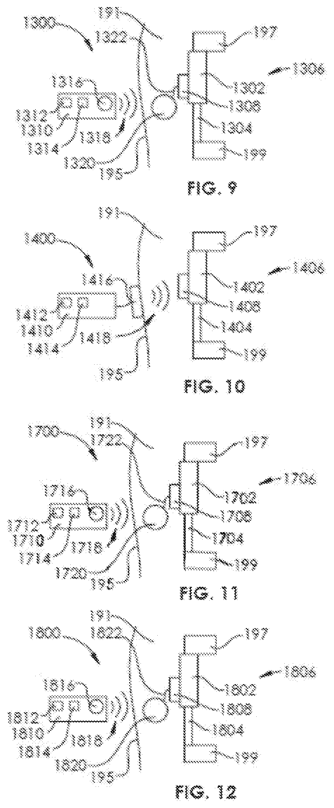

FIG. 10 illustrates an intramedullary device 1400 comprising an implant 1406 having a first implant portion 1402 and a second implant portion 1404, the second implant portion 1404 being non-invasively displaceable with respect to the first implant portion 1402. The first implant portion 1402 is secured to a first bone portion 197 and the second implant portion 1404 is secured to a second bone portion 199 within a patient 191. An ultrasonic motor 1408 is operable to cause the first implant portion 1402 and the second implant portion 1404 to displace relative to one another (e.g., a piezoelectric actuator). An external adjustment device 1410 has a control panel 1412 for input by an operator, a display 1414 and an ultrasonic transducer 1416 that is coupled to the skin 195 of the patient 191. The ultrasonic transducer 1416 produces ultrasonic waves 1418 which pass through the skin 195 of the patient 191 and operate the ultrasonic motor 1408.

FIG. 11 illustrates an intramedullary device 1700 comprising an implant 1706 having a first implant portion 1702 and a second implant portion 1704, the second implant portion 1704 being non-invasively displaceable with respect to the first implant portion 1702. The first implant portion 1702 is secured to a first bone portion 197 and the second implant portion 1704 is secured to a second bone portion 199 within a patient 191. A shape memory actuator is operable to cause the first implant portion 1702 and the second implant portion 1704 to displace relative to one another. An external adjustment device 1710 has a control panel 1712 for input by an operator, a display 1714 and a transmitter 1716. The transmitter 1716 sends a control signal 1718 through the skin 195 of the patient 191 to an implanted receiver 1720. Implanted receiver 1720 communicates with the shape memory actuator 1708 via a conductor 1722. The shape memory actuator 1708 may be powered by an implantable battery, or may be powered or charged by inductive coupling.

FIG. 12 illustrates an intramedullary device 1800 comprising an implant 1806 having a first implant portion 1802 and a second implant portion 1804, the second implant portion 1804 being non-invasively displaceable with respect to the first implant portion 1802. The first implant portion 1802 is secured to a first bone portion 197 and the second implant portion 1804 is secured to a second bone portion 199 within a patient 191. A hydraulic pump 1808 is operable to cause the first implant portion 1802 and the second implant portion 1804 to displace relative to one another. An external adjustment device 1810 has a control panel 1812 for input by an operator, a display 1814 and a transmitter 1816. The transmitter 1816 sends a control signal 1818 through the skin 195 of the patient 191 to an implanted receiver 1820. Implanted receiver 1820 communicates with the hydraulic pump 1808 via a conductor 1822. The hydraulic pump 1808 may be powered by an implantable battery, or may be powered or charged by inductive coupling. The hydraulic pump 1808 may alternatively be replaced by a pneumatic pump.

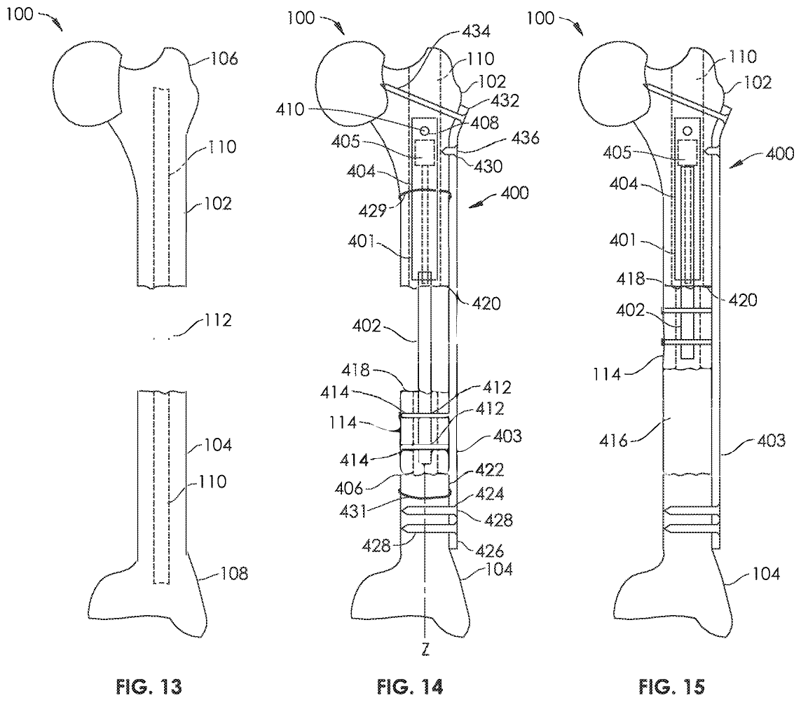

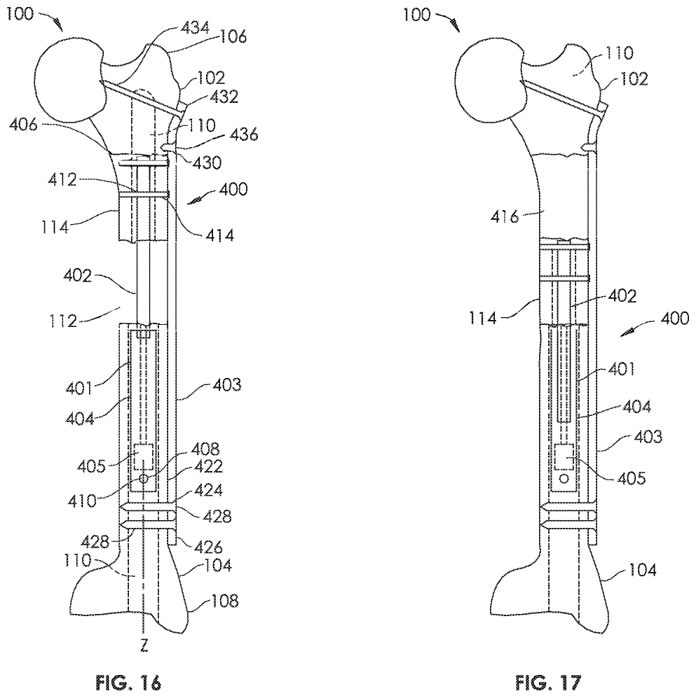

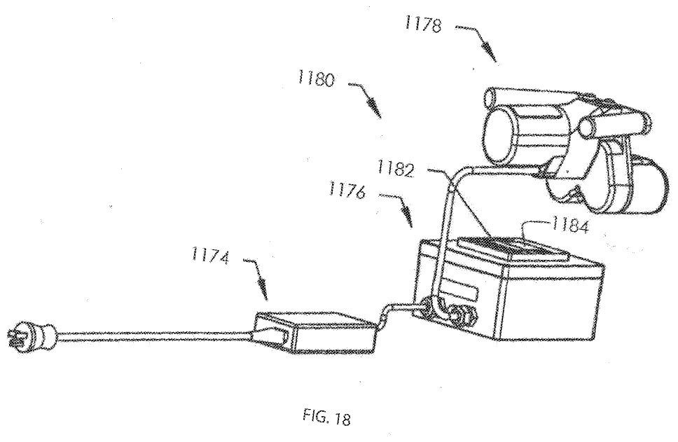

FIG. 13 illustrates a bone 100 which is incomplete and missing a portion. The bone 100 includes a proximal portion 102 and a distal portion 104. The bone 100 has a proximal end 106 and a distal end 108, and a medullary canal 110 extending between the two. The bone may represent a number of different long bones, for example, a femur, a tibia, a fibula, a humerus, or others, or even other bones (e.g., a mandible). An open area 112 between the proximal portion 102 and the distal portion 104 represents the missing bone. The open area 112 may exist for any of a number of reasons. For example, that portion of the bone 100 may have been lost during a traumatic accident or during one or more surgical procedures after a traumatic accident. Or, it may have been removed along with the resection of a portion of cancerous bone, for example, a tumor caused by one or more types of sarcoma.

In FIG. 14, a system for bone transport 400 is shown attached to the bone 100. The system for bone transport comprises an adjustable-length implant 401 and a support member 403. The adjustable-length implant 401 may in some embodiments comprise an intramedullary limb lengthening device, such as the intramedullary device 300 of FIGS. 1-8 or any embodiments shown in FIGS. 9-12. The adjustable implant 401 comprises a rod 402 which is telescopically displaceable from a housing 404. The rod 402 may be distracted out of or retracted into the housing 404 by a driving element 405. In use, the adjustable-length implant 401 may be implanted within the medullary canal 110 of the bone 100 after the medullary canal 110 has been drilled or reamed to remove material or to increase its inner diameter. Prior to or following the implantation of the adjustable-length implant 401, an osteotomy 406 can be made, by cutting, sawing, etc., to create a transport portion 114 of the bone 100. In FIG. 14, the transport portion 114 is created from the distal portion 104 of the bone 100. In other cases, the transport portion 114 may be made from the proximal portion 102 of the bone 100. In FIG. 14, the adjustable-length implant 401 is inserted from the proximal end 106 of the bone 100 (i.e., in an antegrade manner). But, in other cases, the adjustable-length implant 401 may be inserted from the distal end 108 (i.e., in a retrograde manner). With the transport portion 114 separated from the distal portion 104 of the bone 100 by the osteotomy 406. The transport portion 114 and the proximal portion 102 may be coupled to the adjustable-length implant 401 in order to move the transport portion 114 with respect to the proximal portion 102 and distal portion 104. To attach the pieces of the bone 100, the proximal portion 102 of the bone 100 may be drilled on an axis through one or more holes 410 in the housing 404 and one or more bone screws 408 are placed through the one or more holes 410 and secured to the proximal portion 102 of the bone 100. The transport portion 114 of the bone 100 may be drilled on an axis through one or more holes 412 in the rod 402 and one or more bone screws 414 can be placed through the one or more holes 412 and secured to the transport portion 114 of the bone 100. The transport portion 114 may then be non-invasively moved along a longitudinal axis Z of the adjustable-length implant 401. The adjustable-length implant 401 as depicted in FIG. 14 may be supplied to the user in a fully or mostly extended condition (with the rod 402 fully or substantially distracted from the housing 404), so that the transport process moves the transport portion 114 away from the distal portion 104 and towards the proximal portion 102. In this traction manner, the transport portion 114 is pulled not pushed. Pulling on the transport portion 114 tends to provide increased dimensional stability and less drift as the transport portion 114 is being moved. Once a callus begins to acceptably form at the osteotomy 406, the transport process may be started. For example, the transport portion 114 may be moved between about 0.5 mm per day and about 1.50 mm per day, or between about 0.75 mm per day and about 1.25 mm per day, or around 1.00 mm per day. Each daily distraction amount may be achieved in one non-invasive adjustment per day, or may be broken up into two, three, or more separate adjustments (for example, three adjustments of about 0.33 mm each). Due to the osteogenesis that can occur during controlled transport of the transport portion 114, a new bone portion 416 is created. When the bone transport proceeds to the extent such that a proximal end 418 of the transport portion 114 reaches a distal end 420 of the proximal portion 102, a compressive force may be applied to the transport portion 114 and the proximal portion 102. Such compressive forces can help fuse or adhere the transport portion 114 to the proximal portion, and is aided by the fact that it is being applied by pulling the transport portion 114.

As mentioned above, the system for bone transport 400 may also include a support member 403, which may comprise a bone plate configured to be secured to a location on an external surface 422 of the bone 100. The bone plate may comprise a cortical bone plate. The support member 403 may include one or more holes 424 at its distal end 426 for placement of one or more bone screws 428. The support member 403 may also include one or more holes 430 at its proximal end 432 for placement of one or more bone screws 434, 436. The bone screws 434, 428 may be bicortical bone screws and the bone screw 436 may be a unicortical bone screw. Bicortical bone screws may advantageously be used at locations on the bone 100 that are proximal or distal to the adjustable-length implant 401, while unicortical bone screws may advantageously be used at locations on the bone 100 that are adjacent the adjustable-length implant 401. The bone screws 428, 434, 436 that are used to secure the support member 403 to the bone 100 may have threaded shafts and tapered, threaded heads that are configured such that the threaded shafts engage with bone material and the tapered threaded heads engage with tapered threaded holes (e.g., the one or more holes 424, 430) in the support member 403. The support member 403 maintains the proximal portion 102 and the distal portion 104 of the bone 100 static and stable with respect to each other, thereby optimizing the precision of movement of the transport portion 114 as it is moved in relation to the proximal portion 102 and the distal portion 104. One or more cerclages 429, 431 may be used to further secure the system in place, for example, to further secure the support member 403 to the bone 100. While the cerclages 429, 431 are omitted in FIG. 15, it should be understood that they may be used with any embodiment of apparatus or methods described herein. In some embodiments, the support member 403 may include considerably more holes for placement of bone screws. For example, a portion of the support member 403 configured to be placed at the proximal end of a femur may have three, four, or more holes for placement of bone screws which are configured to be secured into bone and extend into the femoral neck, the greater trochanter, or other portions of the femur, including one or more bone fragments.

FIGS. 16 and 17 illustrate the system for bone transport 400 secured to the bone 100. The adjustable-length implant 401, however, has been inserted into the medullary canal 110 from the distal end 108 of the bone (i.e., in a retrograde manner). The osteotomy 406 is thus made in the proximal portion 102 of the bone 100, and the transport portion 114 is detached from the proximal portion 102 of the bone. The transport portion 114 is transported away from the proximal portion 102 of the bone 100 and towards the distal portion 104 of the bone 100, to create the new bone portion 416.

An alternative anatomical setup may be created during surgery, by placing the adjustable-length implant 401 in an orientation similar to that of FIG. 14 (e.g., rod 402 extending distally or oriented downward and housing 404 extending proximally or oriented upward), but by inserting it retrograde (i.e., from the distal end 108 of the bone 100) as shown in FIG. 16. Still another alternative anatomical setup may be created in surgery, by placing the adjustable-length implant 401 in an orientation similar to that of FIG. 16 (e.g., rod 402 extending proximally or oriented upward and housing 404 extending distally or oriented downward), but by inserting it antegrade (i.e., from the proximal end 106 of the bone 100) as shown in FIG. 14.

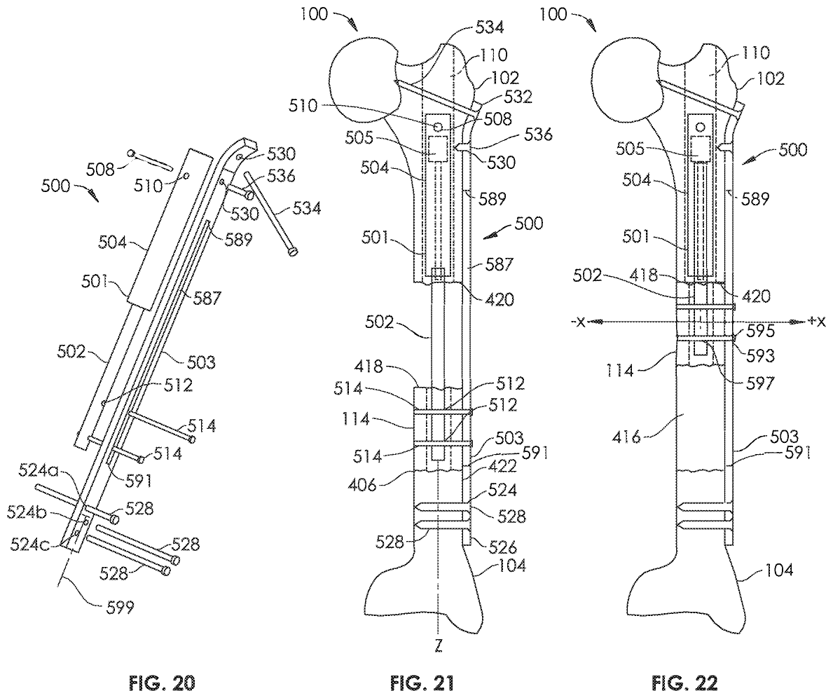

FIG. 20 illustrates a system for bone transport 500. The system for bone transport comprises an adjustable-length implant 501 and a support member 503 (for example, a plate). The adjustable-length implant 501 may in some embodiments comprise an intramedullary limb lengthening device, such as the intramedullary device 300 of FIGS. 1-8 or any of the alternative embodiments of FIGS. 9-12. The adjustable implant 501 may comprise a rod 502, which is telescopically displaceable from a housing 504. The rod 502 may be distracted out of or retracted into the housing 504 by a driving element 505 (shown in FIGS. 21-22). In use, the adjustable-length implant 501 is implanted within the medullary canal 110 of the bone 100, after the medullary canal 110 has been drilled or reamed, to remove material or to increase its inner diameter. Prior to or following this, an osteotomy 406 is made, by cutting, sawing, etc., to create a transport portion 114 of the bone 10. In FIG. 21, the transport portion 114 is created from the distal portion 104 of the bone 100. In other cases, the transport portion 114 may be made from the proximal portion 102 of the bone 100. FIG. 21 illustrates the adjustable-length implant 501 after having been inserted in an antegrade manner. But in other cases the adjustable-length implant 501 may be inserted in a retrograde manner. After separation of the transport portion 114 from the distal portion 104 of the bone 100 (e.g., by the osteotomy 406), the transport portion 114 and the proximal portion 102 may be coupled to the adjustable-length implant 501 in order to move the transport portion 114 with respect to the proximal portion 102 and distal portion 104. The proximal portion 102 of the bone 100 may be drilled on an axis through one or more holes 510 in the housing 504 and one or more bone screws 508 may be placed through the one or more holes 510 and secured to the proximal portion 102 of the bone 100. The transport portion 114 of the bone 100 may be drilled on an axis through one or more holes 512 in the rod 502 and one or more bone screws 514 may be placed through the one or more holes 512 and secured to the transport portion 114 of the bone 100. The transport portion 114 may then be non-invasively moved along a longitudinal axis Z of the adjustable-length implant 501. The adjustable-length implant 501 as depicted in FIG. 21 may be supplied to the user in a fully or mostly extended condition (with the rod 502 fully or substantially distracted from the housing 504), so that the transport process moves the transport portion 114 away from the distal portion 104 and towards the proximal portion 102 In this traction manner, the transport portion 114 is pulled not pushed. Pulling on the transport portion 114 tends to provide increased dimensional stability and less drift as the transport portion 114 is being moved. The support member 503 is similar to the support member 403 of FIGS. 14-17, except that the support member 503 comprises a longitudinal slot 587 extending between a proximal slot end 589 and a distal slot end 597. The slot 587 is located between the proximal end 532 and the distal end 526 of the support member 503. As in the embodiments shown in FIGS. 14-17, the support member 502 may be secured to the bone 100 with one or more bicortical bone screws 528, 534 (which can be placed through holes 524, 530) and one or more unicortical bone screws 536 (which are placed through holes 524, 530). As shown in FIG. 20, certain holes 524 a. 524 c may be offset to one side of centerline 599 of the support member 503, while other holes 524 b, may be offset to another side of centerline 599 of the support member 503. Offsetting the holes in this fashion may aid the placement of bicortical bone screws, in cases wherein the adjustable-length implant 501 extends to the level of the holes 524 a-c. The offset location of the holes 524 a-c, for example, may allow the bicortical bone screws to extend past the rod 502 on either side of the rod 502. The transport portion 114 of the bone 100 can be secured to the rod 502 by the bone screws 514 by drilling the bone 100 in the transport portion along the axes of the holes 512 in a manner such that when the bone screws 514 are secured, they extend from an external location 593 of the slot 587 of the support member 503, through the slot 587, and into the bone 100 of the transport portion 114. The bone screws 514 are aligned in a manner such that when the rod 502 is non-invasively translated with respect to the housing 504, the shaft 597 of the bone screws 514 slide within the slot 587. As will be readily appreciated, the diameter of the shaft 597 of the bone screw 514 is less than the width of the slot 587. In some embodiments, the diameter of the head 595 of the bone screw 514 is greater than the width of the slot 587, thereby further stabilizing the transport portion 114 and limiting its ability to displace in along an x-axis. Turning to FIG. 22, the transport portion 114 itself is limited by the support member 503 so that the transport portion 114 does not translate (drift) substantially in the positive x direction. The transport portion 114 may also be limited by the head 595 of the bone screw 514 so that the transport portion 114 does not translate substantially in the negative x direction, either during longitudinal adjustment of the transport portion, or when at rest.

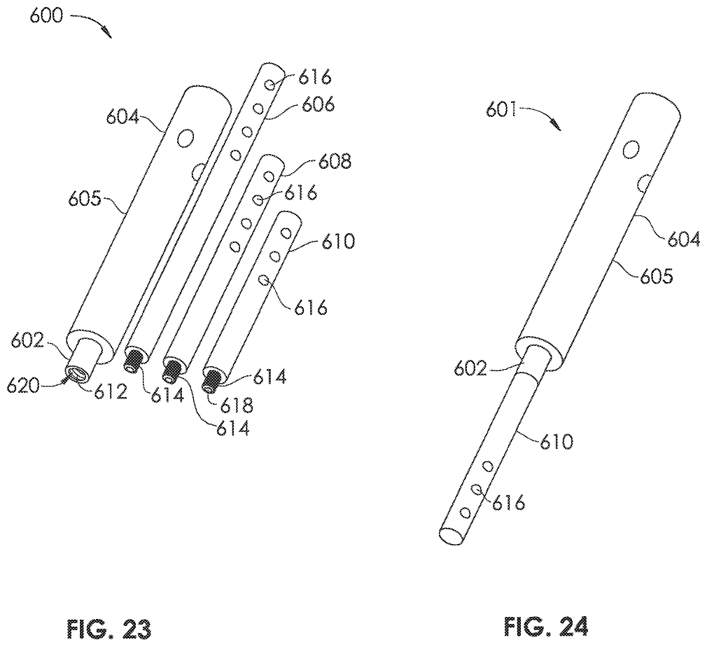

In bone transport or limb lengthening, the transport or distraction lengths can vary greatly from procedure to procedure and/or patient to patient. In bone transport procedures, the transport length may be a function of the length of bone that is missing and the length of the transport portion 114 created during surgery. An adjustable-length implant kit 600 (shown in in FIG. 23) may be configured to allow the user to create an adjustable-length implant, for example the adjustable-length implant 601 of FIG. 24, tailored to the particular transport length or distraction length of the patient to be treated. The adjustable-length implant kit 600 may include a base actuator 605 comprising a housing 604, a base rod 602, and one or more rod extensions (e.g., rod extensions 606, 608, 610). The base rod 602 may be telescopically moveable within the housing 604 (as described elsewhere herein) and has an internally threaded portion 612. Each of the rod extensions 606, 608, 610 has an externally threaded portion 614 which is configured to be screwed into the internally threaded portion 612 of the base rod 602. A user (e.g., surgeon or physician) may choose the appropriate rod extension 606, 608, 610 for the particular patient. For example, rod extension 606 may be chosen if a relatively long transport or distraction length is required, whereas rod extension 610 may be chosen if a relatively short transport or distraction length is required. It will be understood that the rod extensions 606, 608, 610 may have varying properties, including but not limited to: numbers of anchor holes 616; axial orientation of anchor holes 616; anchor hole diameters (e.g., for use with bone screw of different diameters); etc. The rod extensions 606, 608, 610 may include a hollow portion. For example, an interior passage 618 may pass through the end of the rod extension 610 (or any other rod extension 606, 608) which has the externally threaded portion 614. In that way, the lead screw (not shown) may extend into the interior passage 618, e.g., if the lead screw extends from the interior of the base rod 602. In some embodiments, the lead screw may be extendible (i.e., may have an end that may be augmented by an extension portion of lead screw). The internally threaded portion 612 and the externally threaded portion 614 may each have a locking feature, incorporating, for example, a latch, snap, detent, hook, or friction fit feature that secures the rod extension 606, 608, 610 and the base rod 602 when the rod extension 606, 608, 610 to the base rod 602 are coupled (e.g., screwed together). In an alternative embodiment, the base rod 602 may include an externally threaded portion and the rod extensions 606, 608, 610 may each include an internally threaded portion. The adjustable-length implant kit 600 of FIGS. 23-24 may be used in standard limb lengthening procedures, or in bone transport procedures, including, but not limited to, those described herein. By having the adjustable-length implant kit 600 available during surgery, a surgeon or physician may more easily select and/or construct a device most appropriate for the patient being treated. In some embodiments, the rod extensions 606, 608, 610 may be easily sterilized (e.g., steam sterilization/autoclave, gas) which may lower the cost of the procedure, especially if the base actuator 605 must be supplied sterile by the supplier. In use, a surgeon or physician (which should be understood to include any other medical professional, such as those under the control or direction of a surgeon or physician) may attach one rod extension, and remove it and replace it with another, if it does not fit the patient properly. In alternative embodiments and methods, the support member 403, 503 may be replaced by an external fixator comprising a base which is configured to be located external to the patient, a first pin configured to attach at one end to the base and at another end to be coupled to the first portion of the bone, and a second pin configured to attach at one end to the base and at another end be coupled to the second portion of the bone.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. In addition, while a number of variations of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. Accordingly, it should be understood that various features and aspects of the disclosed embodiments can be combined with or substituted for one another in order to form varying modes of the disclosed invention. Thus, it is intended that the scope of the present invention herein disclosed should not be limited by the particular disclosed embodiments described above, but should be determined only by a fair reading of the claims that follow.

Similarly, this method of disclosure, is not to be interpreted as reflecting an intention that any claim require more features than are expressly recited in that claim. Rather, as the following claims reflect, inventive aspects lie in a combination of fewer than all features of any single foregoing disclosed embodiment. Thus, the claims following the Detailed Description are hereby expressly incorporated into this Detailed Description, with each claim standing on its own as a separate embodiment.

What is claimed is:

1. A method for bone transport comprising:
providing a system including an adjustable length implant and a support member;
coupling a housing of the adjustable length implant to a first bone portion;
coupling a rod of the adjustable length housing to a transport bone portion to be disposed between the first bone portion and a second bone portion, wherein the rod is moveable relative to the housing;
fixedly coupling a first end of the support member having a to the first bone portion;
fixedly coupling a second end of the support member to a second bone portion,
moving the rod to move the transport bone portion in a first direction relative to the first bone portion and in a second direction opposite the first direction relative to the second bone portion while the support member is fixedly attached to both the first bone portion and the second bone portion, and
wherein the transport bone portion and the first bone portion are initially discrete sections of bone, and wherein the transport bone portion is initially created by performing an osteotomy and cutting a bone section away from the second bone portion prior to moving the transport bone portion towards the first bone portion.

2. The method of claim 1, wherein the support member is configured to maintain a position of the first bone portion relative to the second bone portion while the rod moves the transport bone portion relative to the first bone portion and the second bone portion.

3. The method of claim 1, wherein the system further includes a driving element configured to be non-invasively actuated to displace the rod relative to the housing.

4. The method of claim 3, wherein the driving element comprises a permanent magnet.

5. The method of claim 4, wherein the permanent magnet comprises a radially poled rare earth magnet.

6. The method of claim 1, wherein the support member comprises one or more holes at one or more of its distal end and proximal end, the one or more holes each configured to pass a bone anchor.

7. The method of claim 6, wherein the one or more holes each have a female thread, configured to engage a male thread carried by a head of a bone anchor.

8. The method of claim 3, wherein the adjustable length implant is configured such that when the driving element is non-invasively activated, the distance between the first end and the second end of the adjustable length implant is controllably shortened.

9. The method of claim 1, wherein the system further includes an external adjustment device configured to cause displacement of the rod relative to the housing.

10. A method for bone transport comprising:
providing a system including an adjustable length implant, a support member, and an external adjustment device configured to cause displacement of the rod relative to the housing;
coupling a housing of the adjustable length implant to a first bone portion;
coupling a rod of the adjustable length housing to a transport bone portion to be disposed between the first bone portion and a second bone portion, wherein the rod is moveable relative to the housing;
fixedly coupling a first end of the support member having a to the first bone portion;
fixedly coupling a second end of the support member to a second bone portion,
moving the rod to move the transport bone portion in a first direction relative to the first bone portion and in a second direction opposite the first direction relative to the second bone portion while the support member is fixedly attached to both the first bone portion and the second bone portion, and
wherein the transport bone portion and the first bone portion are initially discrete sections of bone, and wherein the transport bone portion is initially created by performing an osteotomy and cutting a bone section away from the second bone portion prior to moving the transport bone portion towards the first bone portion.

11. The method of claim 1, wherein the rod is further configured to move with the transport bone portion relative to the first end of the support member in a first direction and relative to the second end of the support member in a second direction opposite the first direction.

12. The method of claim 1, wherein the housing is configured to be coupled to the first bone portion through one or more holes in the housing and one or more bone anchors placed through the one or more holes along a longitudinal axis of the adjustable length implant.

13. The method of claim 1, wherein the rod is configured to be coupled to the transport bone portion through one or more holes in the rod and one or more bone anchors placed through the one or more holes along a longitudinal axis of the adjustable length implant.

14. The method of claim 1, wherein in a first configuration of the system, the transport bone portion is adjacent the second bone portion, and wherein in a second configuration of the system, the transport bone portion is pulled by the rod and moves away from the second bone portion and towards the first bone portion.

15. The method of claim 14, wherein a third configuration of the system, the transport bone portion contacts the first bone portion so that both the transport bone portion and the first bone portion receives a compressive force formed by pulling the transport bone portion by the rod.

16. The method of claim 1, wherein the adjustable length implant further includes a rod extension, wherein the rod extension includes an externally threaded portion configured to engage an internally threaded portion of the rod.
